(19) United States
(12) Patent Application Publication
(10) Pub. No.: US 2005/0119190 A1
(43) Pub. Date: Jun. 2, 2005

(54) Inhibitors of the blood-clotting factor xa, production thereof and use of the same

(76) Inventors: Jorg Sturzebecher, Erfurt (DE); Torsten Steinmetzer, Jena (DE); Andrea Schweinitz, Jena (DE)

Correspondence Address: CLARK & ELBING LLP, 101 FEDERAL STREET, BOSTON, MA, 02110, US

(21) Appl. No.: 10/506,383

(22) PCT Filed: Mar. 11, 2003
(86) PCT No.: PCT/EP03/02487

(30) Foreign Application Priority Data
Mar. 11, 2002 (DE) 10210590.1

(51) Int. Cl. A61K 38/04; C07K 5/04

(52) U.S. Cl. 514/19; 564/86; 548/339.1; 548/495; 546/332

(57) ABSTRACT

The invention relates to novel inhibitors of clotting factor Xa, the preparation and use thereof for therapy, prophylaxis and diagnosis of cardiovascular disorders and thromboembolic events.

The invention relates to novel inhibitors of clotting factor Xa, the preparation and use thereof for therapy, prophylaxis and diagnosis of cardiovascular disorders and thromboembolic events.

The heparin-type anticoagulants currently in clinical use, and the vitamin K antagonists do not meet all the requirements for an “ideal” antithrombotic. This is why alternatives are sought in the form of small molecular weight inhibitors of the clotting enzymes, specifically of thrombin and factor Xa (F Xa). A particular advantage of F Xa inhibitors compared with thrombin inhibitors might be the lower tendency to bleeding found in various animal experiments. Thus, there was only a minimal effect on the bleeding time at antithrombotically effective doses (J. M. Herbert et al., J. Pharmacol. Exp. Ther. 276, 1030-1038, 1996; K. Sato et al., Br. J. Pharmacol. 123, 92-96, 1998).

The first non-peptide compounds having high affinity for F Xa were symmetrical bisbenzamidines (Kᵢ=13 nM for the most effective compound BABCH) (J. Stürzebecher et al., Thromb. Res. 54, 245-252, 1998). The naphthamidine derivative DX-9065a also has two basic groups and inhibits F Xa selectively with a Kᵢ=24 nM (T. Hara et al., Thromb. Haemost. 71, 314-319, 1994). The inhibitor YM-60828 (K. Sato et al. Eur. J. Pharmacol. 339, 141-146, 1997) is structurally related to DX-9065a and is even more effective (Kᵢ=1.3 nM). A whole series of further bis-basic compounds in which, for example, two benzamidine residues are linked by an oxazoline ring (Kᵢ=18 nM) (M. L. Quan et al., Bioorg. Med. Chem. Lett. 7, 2813-2818, 1997) or a carboxymethylalkyl chain (Kᵢ=34 nM) (T. P. Maduskuie et al., J. Med. Chem. 41, 53-62, 1998) has now been described. The particular disadvantage of bis-basic compounds is the low bioavailability after oral administration.

F Xa inhibitors containing only one basic group have also been described. N-substituted amidinophenoxypyridines (Kᵢ=0.11 nM for BX-807834) have been developed on the basis of BABCH (R. Mohan et al., Bioorg. Med. Chem. Lett. 8, 1877-1882, 1998; G. B. Phillips et al. J. Med. Chem. 41, 3557-3562, 1998). Amides of Nα-adamantyloxycarbonyl-3-amidinophenylalanine (Kᵢ=74 nM for the most effective compound) are selective inhibitors of F Xa (S. Sperl et al., Biol. Chem. 381, 321-329, 2000), while Nα-arylsulfonylaminoacylated esters of 3-amidinophenylalanine have a small inhibitory effect (Kᵢ=840 nM for TAPAM) (J. Stürzebecher et al., Thromb. Res. 54, 245-252, 1998). WO 96/10022 discloses inhibitors which now have no strong charge at all (Kᵢ=3.0 nM for the most effective compound).

To date, only a few peptides derived from the substrate sequence Ile-Glu-Gly-Arg have been described as inhibitors of F Xa. The chloromethyl ketones described by Kettner and Shaw (Thromb. Res. 22, 645-652, 1981) inhibit F Xa irreversibly and are unsuitable for in vivo applications. By contrast, the peptides SEL 2489 (Kᵢ=25 nM) and SEL 2711 (Kᵢ=3 nM) are extremely effective (J. A. Ostrem et al., Biochemistry 37, 1053-1059, 1998). There have also been descriptions of some peptidyl-arginine aldehydes and peptidyl-arginyl ketones which, besides argininal or an arginyl ketone derivative such as, for example, arginyl-ketothiazole in the P3 position, have a D-arginine or an unnatural basic amino acid such as, for example, 4-amidinophenylalanine, 3- or 4-amidinopiperidinylalanine and 4-guanidinophenylalanine in P3 (Z. H. Jonathan, Bioorg. Med. Lett. 9, 3459-3464, 1999 and review article: Zhu and Scarborough Current Opinion in Cardiovascular, Pulmonary & Renal Investigational Drugs 1999, 1, 63-88).) The application WO 01/96366 discloses inhibitors which are derived from acylated amidinobenzylamine and, besides a natural amino acid in P2, contain a D-Ser ether or a comparable derivative of an unnatural amino acid. Compounds of this type very effectively inhibit both F Xa (Kᵢ=30 nM for the most effective compound) and the clotting of human blood plasma. However, compounds of this type have pharmacokinetic properties which are inadequate for application in vivo; they are scarcely absorbed after oral adminstration and, in experimental animals, are eliminated very rapidly from the circulation after i.v. administration.

The invention is therefore based on the object of indicating an active ingredient which is also suitable for therapeutic applications and which inhibits clotting factor Xa with high activity and specificity and which circulates in the body as long as possible after i.v., s.c. or oral administration.

It has surprisingly been found that an acylated amidinobenzylamine according to the general formula I specified in claim 1 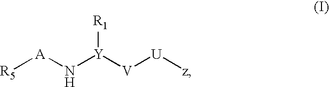
where

A is P₂-P₁ with 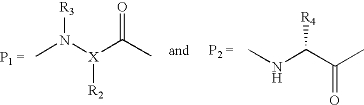
in particular compounds of 4-amidinobenzylamine in which X, R₂, R₃ and R₄ yield natural and/or unnatural amino acids, both very effectively inactivate factor Xa and are slowly eliminated from the circulation on introduction, besides the amidino function, of further charged groups, preferably carboxyl, amino, amidino, hydroxyamidino, amidrazono or guanidino. The carboxyl groups may also be protected in the form of their esters, with ethyl esters preferably being used. These esters are partially converted into the free acids in vivo.

The designation of the residues P₂ and P₁ in the structural segment A of the general formula I does not relate to the otherwise normally used nomenclature of amino acid residues in peptide substrates of serine proteases and inhibitors derived therefrom, as introduced by Schechter and Berger (Schechter and Berger, Biochem. Biophys. Res. Comm. 27, 157-162 (1967)). The definitions applying in all parts of the invention, i.e. both in the description and in the claims, are as follows:

The letter P in connection with a number from 1 to 3 in normal script, i.e. P1, P2 or P3, is used for amino acid residues and derivatives thereof in accordance with the Schechter and Berger nomenclature. By contrast, the letter P associated with a subscript 1 or 2, i.e. P₁ or P₂, stands for amino acid residues and derivatives thereof as constituents of structure A in formula I of the present invention. In this connection, substituted or unsubstituted natural or unnatural amino acid P₁ in the structure A corresponds to P2 according to Schechter and Berger, and the substituted or unsubstituted natural or unnatural amino acid P₂, which is in the D configuration, in structure A corresponds to P3 according to Schechter and Berger.

In formula I

R₁ is an H or —(CH₂)ₐCOOR₆ with a=0, 1, 2, 3, 4 or 5, preferably with a=0, 1 or 2, where R₆ is a branched or unbranched alkyl radical having preferably 1 to 6 C atoms, in particular 1 to 3 C atoms, especially ethyl;

R₂ is an H, a branched or unbranched alkyl radical having 1 to 8 C atoms, preferably having 1 to 3 C atoms, or —(CH₂)_{c}COOR₈ with c=1, 2, 3 or 4, where R₈ is H or a branched or unbranched alkyl radical having preferably 1 to 6 C atoms, in particular 1 to 3 C atoms, especially ethyl, or —(CH₂)_{d}—OR₉ with d=1, 2, 3 or 4, where R₉ is H, or —(CH₂)ₑOR₁₀, —(CH₂)ₑSR₁₀, —(CH₂)ₑ-guanidino, —(CH₂)ₑ-imidazole or —(CH₂)ₑNHR₁₀ with e=1, 2, 3, 4 or 5, where R₁₀ is H, a branched or unbranched alkyl radical having 1-16, in particular 1-8, especially 1-3, C atoms or a substituted or unsubstituted aryl, heteroaryl, aralkyl or heteroaralkyl radical, where the alkyl radical preferably has 1 to 16, in particular 1 to 8, especially 1 to 3, C atoms, and the aryl or heteroaryl radical preferably has 4 to 14, in particular 6 to 10, especially 6, C atoms and preferably 1 to 3 N as heteroatom;

R₃ is an H or —(CH₂)_{b}R₇ with b=1, 2, 3, 4, 5, 6, 7 or 8, preferably with b=2 or 3, where R₇ is H, a branched or unbranched alkyl radical having 1 to 10 C atoms, preferably having 1 to 3 C atoms, or a charged radical, preferably a —(CH₂)ⱼCOOR₁₃, —(CH₂)ⱼSO₂R₁₃, —(CH₂)ⱼNH₂, —(CH₂)ⱼ-amidino, —(CH₂)ⱼ-hydroxyamidino or —(CH₂)ⱼ-guanidino group with j=0, 1 or 2, where R₁₃ is H or an alkyl radical having preferably 1 to 6 C atoms, in particular 1 to 4, especially ethyl;

R₄ is a branched or unbranched alkyl radical having 1 to 8, preferably 1 to 3, C atoms, —(CH₂)_{f}OR₁₁, —(CH₂)_{f}SR₁₁, —(CH₂)_{f}-guanidino, —(CH₂)_{f}-imidazole, —(CH₂)_{f}—R₁₁ or —(CH₂)_{f}NHR₁₁ with f=1, 2, 3, 4 or 5, preferably 1 or 2, in particular 1, where R₁₁ is H, a branched or unbranched alkyl radical having 1 to 16, preferably 1 to 8, in particular 1-4 C atoms, especially tbutyl or a substituted or unsubstituted aryl, heteroaryl, aralkyl or heteroaralkyl radical, where the alkyl radical preferably has 1 to 16, in particular 1 to 8, especially 1 to 3, C atoms, and the aryl or heteroaryl radical preferably has 4 to 14, in particular 6 to 10, especially 6, C atoms and preferably I to 3 N as heteroatom; where P₂ in the structure A of the general formula I is in the D configuration;

R₅ is —(CH₂)_{g}(CH₃)ₕ, —(CH₂)ᵢ-aryl with g+h=i=0, 1, 2 or 3, —SO₂R₁₂, —COR₁₂, or —COOR₁₂, where R₁₂ is a branched or unbranched alkyl having 1-16, preferably 1 to 8, in particular 1-4, especially 1-2, C atoms, a substituted or unsubstituted aryl, heteroaryl, aralkyl or heteroalkyl radical, an adamantyl, a camphor, a cyclohexylmethyl radical, preferably benzyl,

where R₅ may be modified with a charged or uncharged group, preferably a —(CH₂)ⱼCOOR₁₃, —(CH₂)ⱼSO₂R₁₃, —(CH₂)ⱼNH₂, —(CH₂)ⱼ-amidino, —(CH₂)ⱼ-hydroxyamidino or —(CH₂)ⱼ-guanidino group with j=0, 1 or 2, where R₁₃ is H or an alkyl radical having preferably 1 to 6 C atoms, in particular 1 to 4, especially ethyl;

U is a phenyl or cyclohexyl radical; a heterophenyl or heterocyclohexyl radical having preferably at least one N, S or O as heteroatom, in particular pyridine, piperidine or pyrimidine or a thiophene radical;

V is (CH₂)ₙ with n=0, 1, 2 or 3, preferably 0;

X is N or CH, preferably CH;

Y is N or (CH)ₘ with m=0 or 1, preferably CH;

Z occurs in the 3 or 4 position and is an aminomethyl, a guanidino function or an amidino group 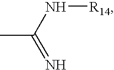
where R₁₄ is H, OH, NH₂, —COR₁₅ or —COOR₁₅, where R₁₅ is a branched or unbranched alkyl radical having 1 to 16, preferably 1 to 8, in particular 1 to 4, especially 1 to 2, C atoms or a substituted or unsubstituted aryl or heteroaryl, aralkyl or heteroaralkyl radical, where the alkyl radical preferably has 1 to 16, in particular 1 to 8, especially 1 to 4 and particularly preferably 1 to 2, C atoms and the aryl or heteroaryl radical preferably has 4 to 14, in particular 6 to 10, especially 6, C atoms and preferably 1 to 3 N as heteroatom;
where one or more charged radicals preferably derived from —COOH, —CH(COOH)₂, —SO₂H, NH₂, an amidino, hydroxyamidino, amidrazono or guanidino group are present in the radicals R₁, R₂, R₃ or R₅;
or a compound of the general formula I in the form of a prodrug or in the form of its salt.

A prodrug for the purposes of the present invention is an acrylated amidino- or guanidinobenzylamine of the general formula I, which is in the form of a pharmaceutically inactive derivative of the appropriate pharmaceutically active substance and, after oral administration, is spontaneously or enzymatically biotransformed to liberate the pharmaceutically active substances.

Further particularly suitable compounds are compounds of the formula I where U is substituted at 1, 2 or 3 positions preferably by a halogen, in particular fluorine or chlorine, or a methyl, ethyl, propyl, methoxy, ethoxy or propoxy radical.

Further particularly suitable compounds are compounds of the general formula I where a carboxyl group is present protected as ester, preferably as ethyl ester, and is converted into a carboxyl group in the manner of a prodrug only after intake in the body.

Further particularly suitable compounds are compounds of the general formula I where R₉ in this case is an alkylcarbonyl, aralkylcarbonyl, alkyloxycarbonyl or aralkyloxycarbonyl radical, where the alkyl radical preferably has 1 to 6, in particular 1 to 4, C atoms and the aryl radical preferably has 5 to 8, in particular 6, C atoms; and where R₉ is converted into a carboxyl group in the manner of a prodrug only after intake in the body.

Further particularly suitable compounds are compounds of the general formula I where P₂ in structure A of the general formula I is derived from one of the following amino acids in the D configuration: D-2,3-diaminopropionic acid, D-2,4-diaminobutyric acid, D-ornithine, D-arginine, D-homoarginine, D-norarginine, D-4-guanidinophenylalanine, D-4-guanidinophenylhomoalanine, D-4-guanidinophenylglycine, D-3-guanidinophenylalanine, D-3-guanidinophenylhomoalanine, D-3-guanidinophenylglycine, D-4-amidinophenylalanine, D-4-amidinophenylhomoalanine, D-4-amidinophenylglycine, D-3-amidinophenylalanine, D-3-amidinophenylhomoalanine, D-3-amidinophenylglycine, D-4-aminomethylphenylalanine, D-4-aminomethylphenylhomoalanine, D-4-aminomethylphenylglycine, D-3-aminomethylphenylalanine, D-3-aminomethylphenylhomoalanine, D-3-aminomethylphenylglycine, D-4-aminophenylalanine, D-4-aminophenylhomoalanine, D-4-aminophenylglycine, D-3-aminophenylalanine, D-3-aminophenylhomoalanine, D-3-aminophenylglycine, D-4-guanidinomethylphenylalanine, D-4-guanidinomethylphenylhomoalanine, D-4-guanidinomethylphenylglycine, 3-guanidinomethylphenylalanine, D-3-guanidinomethylphenylhomoalanine, D-3-guanidinomethylphenylglycine, D-4-piperidinylalanine, D-4-piperidinylhomoalanine, D-4-piperidinylglycine, D-4-N-(amidino)piperidinylalanine, D-4-N-(amidino)piperidinylhomoalanine, D-4-N-(amidino)piperidinylglycine, D-3-piperidinylalanine, D-3-piperidinylhomoalanine, D-3-piperidinylglycine, D-3-amidinopiperidinylalanine, D-3-amidinopiperidinylhomoalanine, D-3-amidinopiperidinylglycine, D-4-aminocyclohexylalanine in cis or trans, D-4-aminocyclohexylhomoalanine in cis or trans, D-4-aminocyclohexylglycine in cis or trans, n-butylamidinoglycine, n-pentylamidinoglycine or n-propylamidinoglycine. D-Alanine(3-(1-N-piperazinyl) or D-homoalanine(3-(1-N-piperazinyl). The common feature of said amino acids is that they are arginine derivatives.

Further particularly preferred compounds are compounds of the general formula I where P₂ in the structure A of the general formula I is likewise derived from an amino acid in the D configuration, which is an arginine derivative but which has a lower basicity than the amino acids mentioned in the previous paragraph. Particularly suitable examples thereof are: D-canavanine, D-homocanavanine, D-norcanavanine; D-canavanine is synthesized in analogy to the method for L-canavanine with D-homoserine as starting material (Kim et al., Med. Chem. Res. 377-383 (1996). Further examples are: 2-amino-4-amidinohydrazonobutyric acid, 2-amino-5-amidinohydrazonopropionic acid, 2-amino-5-amidinohydrazonopentanoic acid. Synthesis takes place according to the strategy described for introducing the amidrazono group into a series of thrombin inhibitors (Soll et al., Bioorg. Med. Chem. Lett. 10 (2000) 1-4). In addition 2-amino-4-(pyridin-4-ylamino)butyric acid, 2-amino-4-(pyridin-4-ylamino)propionic acid, 2-amino-4-(pyridin-4-ylamino)pentanoic acid, where the synthesis of the aminopyridine derivatives takes place as described in: von der Saal, Bioorg. Med. Chem. Lett. 7, 1283-1288 (1997). A further example is 4-imidazolylpropargylglycine, which is prepared from propargylglycine (Advanched Chemtech) and Pd-catalyzed coupling with N-trityl-4-iodoimidazole in analogy to the following references: Lee et al., Bioorg. Med. Chem. Lett. 10, 2775-2778 (2000); Kirk, K. I. J. Heterocycl. Chem. 22, 57 ff. (1985). Further examples are: D-histidine, D-homohistidine, D-histidine-(1-methyl), D-homohistidine-(1-methyl), D-histidine-(3-methyl), D-homohistidine-(3-methyl), D-alanine(4-[5-2(-amino)imidazoyl], D-homoalanine(4-[5-2(-amino)imidazoyl], D-glycine(4-[5-2(-amino)imidazoyl], D-alanine(4-pyridyl), D-homoalanine(4-pyridyl), D-glycine(4-pyridyl), D-alanine(3-pyridyl), D-homoalanine(3-pyridyl), D-glycine(3-pyridyl), D-alanine(2-pyridyl), D-homoalanine(2-pyridyl), D-glycine(2-pyridyl), D-alanine(3-(2-pyrimidinyl), D-homoalanine(3-(2-pyrimidinyl), D-alanine(3-(5-pyrimidinyl), D-homoalanine(3-(5-pyrimidinyl), D-2-amino-3-(2-aminopyrimidin-5-yl)propionic acid, D-2-amino-4-(2-amino-pyrimidin-5-yl)butyric acid, D-alanine(3-(2-benzimidazolyl)), D-homoalanine(3-(2-benzimidazolyl)), D-alanine(3-(3-quinolinyl), D-homoalanine(3-(3-quinolinyl), D-tryptophan, D-homotryptophan, D-tryptophan substituted by aminoalkyl groups on the indole ring, D-homotryptophan substituted by aminoalkyl groups on the indole ring, D-2-amino-3-(6-aminopyridin-3-yl)propionic acid, D-2-amino-4-(6-aminopyridin-3-yl)butyric acid, D-2-amino-3-(6-amino-2-methylpyridin-3-yl)-propionic acid, D-2-amino-4-(6-amino-2-methylpyridin-3-yl)butyric acid, D-2-amino-3-(6-amino-2,4-dimethylpyridin-3-yl)propionic acid, D-2-amino-4-(6-amino-2,4-dimethylpyridin-3-yl)butyric acid, D-citrulline, D-homocitrulline, D-norcitrulline, D-4-hydroxyamidinophenylalanine, D-4-hydroxyamidinophenylhomoalanine, D-4-hydroxyamidinophenylglycine, D-3-hydroxyamidinophenylalanine, D-3-hydroxyamidinophenylhomoalanine or D-3-hydroxyamidinophenylglycine. An advantage of factor Xa inhibitors with these less basic D-arginine mimetics is that they are only partly charged at physiological pH and therefore oral uptake is better.

Further particularly suitable compounds are compounds of the general formula I, where the compound has the following structure 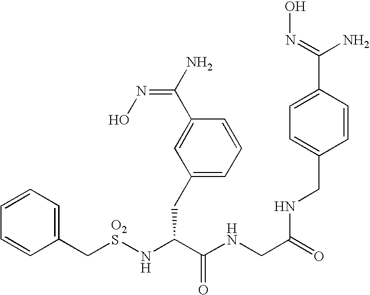
where the hydroxyamidino groups present in the structure are converted into the analogous amidino groups in the manner of a prodrug only after intake in the body, resulting in the inhibitor structure with inhibitory activity.

Further particularly suitable compounds are compounds of the general formula I where the substituent on the substituted aryl, heteroaryl, aralkyl or heteroaralkyl radical is a halogen, preferably fluorine, chlorine or bromine, in particular fluorine or chlorine.

Besides inactivation of factor Xa, the additionally charged 4-amidinobenzylamine derivatives are in an advantageous and surprising manner eliminated very slowly, so that the compounds of the invention represent a novel group of highly active F Xa inhibitors.

The compounds are usually in the form of salts, preferably with mineral acids, preferably as hydrochlorides, or preferably as salts with suitable organic acids. Preferred salts of mineral acids are also sulfates. Examples of suitable organic acids are acetic acid, formic acid, methylsulfonic acid, succinic acid, malic acid or trifluoroacetic acid, with preferred salts of organic acids being acetates.

The compounds of the general formula I can be prepared in a manner known in principle as described below, for example as follows:

from the commercially available 4-cyanobenzylamine (Showa Denko, Japan), the Boc-protected 4-acetyloxamidinobenzylamine is obtained by methods known to the skilled worker. Elimination of the Boc-protective group is followed by coupling on the further amino acids and the protective group R₅ by means of standard coupling methods with Boc as N-terminal protective group. The second amino acid can also be coupled directly as N-arylsulfonyl- or N-aralkylsulfonyl-protected amino acid. The peptide analogs are assembled sequentially starting from acetyloxamidinobenzylamine. Most of the intermediates crystallize well and can thus be purified easily. Final purification of the inhibitors takes place at the last stage preferably by preparative reversed phase HPLC.

The invention further relates to a method for preparing a compound of the general formula I, where the appropriate amino acids are coupled sequentially onto a 4-acetyloxamidinobenzylamine, with the N-terminal amino acid either already carrying the R₅ radical or the latter subsequently being linked thereto.

The invention further relates to a medicament comprising an inhibitor of the invention, and further pharmaceutically suitable excipients and/or additives. Suitable excipients and/or additives, which serve for example to stabilize and/or preserve the medicament, are generally familiar to the skilled worker (e.g. Sucker H. et al., (1991) Pharmazeutische Technologie, 2nd edition, Georg Thieme Verlag, Stuttgart). These include, for example, physiological saline solutions, Ringer dextrose, Ringer lactate, demineralized water, stabilizers, antioxidants, complexing agents, antimicrobial compounds, proteinase inhibitors and/or inert gases.

The medicament could for example be used in parenteral use form, in particular in intraarterial, intravenous, intramuscular or subcutaneous form, in an enteral use form, in particular for oral or rectal use, or in a topical use form, in particular as dermatologic agent. Intravenous or subcutaneous uses are preferred.

In one embodiment of the invention, the medicament is employed for example in the form of a tablet, of a coated tablet, of a capsule, of a pellet, suppository, of a solution, in particular of a solution for injection or infusion, of eyedrops, nose and eardrops, of a syrup, of a capsule, of an emulsion or suspension, of a pessary, stick, aerosol, dusting powder, of a paste, cream or ointment.

The factor Xa inhibitors of the invention or the medicaments mentioned are preferably used for the diagnosis, therapy or prophylaxis of a cardiovascular disorder or of a thromboembolic event, in particular in oral, subcutaneous, intravenous or transdermal form.

The invention is to be explained in more detail below by means of three exemplary embodiments without restricting it.

Methods

Analytical HPLC: Shimadzu LC-10A system, column: Vydac C₁₈, 5 μm (250×4 mm) solvents A: 0.1% TFA in water, B: 0.1% TFA in ACN, gradient: 10% B to 60% B in 50 min, 1 ml/min flow rate, detection at 220 or 215 nm. Preparative HPLC: Shimadzu LC-8A System, column: Knauer C₁₈, 5 μm (250×32 mm) solvents A: 0.1% TFA in water, B: 0.1% TFA in ACN, gradient: 10% B to 55% B in 120 min, 10 ml/min flow rate, detection at 220 nm. Mass spectroscopy: The mass spectra were recorded on a Kompact Probe from Kratos (Manchester, England) with a time of flight measuring detector and α-cyanohydroxycinnamic acid as matrix, or on an ESI-MS LCQ from Finnigan (Bremen, Germany).

### EXAMPLE 1

### Synthesis of Benzylsulfonyl-D-Ser(tBu)-Glu 4-amidinobenzylamide×TFA

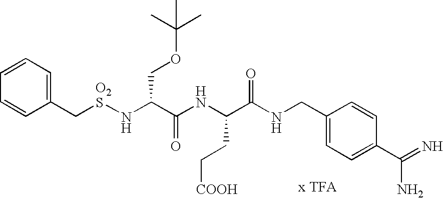

1.1 Boc-4-cyanobenzylamide

20 g (0.151 mol) of 4-cyanobenzylamine were dissolved in 300 ml of H₂O, 150 ml of dioxane and 150 ml of 1 N NaOH. While cooling in ice, 37.5 ml of di-tert-butyl dicarbonate were added dropwise, and the mixture was stirred at 0° C. for one hour and at room temperature for a further 24 h. The dioxane was removed in vacuo, and the product was taken up in ethyl acetate and 5% KHSO₄ solution. The ethyl acetate phase was washed 3 times with 5% KHSO₄ solution and 3 times with saturated NaCl solution, dried over Na₂SO₄ and concentrated in vacuo (white crystals). HPLC: acetonitrile/H₂O, elution at 44.1% acetonitrile; yield: 30.48 g (0.131 mol), 87%.

1.2 Boc-4-acetyloxamidinobenzylamide

In accordance with Judkins et al. (Synthetic Comm. 26, 4351-4367, 1996), 30.48 g (0.131 mol) of Boc-4-cyanobenzylamide were dissolved with 13.65 g (0.197 mol) of hydroxylamine×HCl and 34 ml (0.197 mol) of DIEA in 300 ml of abs. ethanol. The mixture was boiled under reflux for 2 h and stirred at room temperature overnight. The mixture was then concentrated in vacuo, and the residue was dissolved in approx. 200 ml of acetic acid, and 18.67 ml (0.197 mol) of acetic anhydride were added. After 1 h, the mixture was again concentrated, dissolved in ethyl acetate and, at 0° C., washed 3 times each with 5% KHSO₄ solution and saturated NaCl solution. Drying over Na₂SO₄ and concentration in vacuo resulted in a white powder. HPLC: acetonitrile/H₂O, elution at 32.0% acetonitrile; yield: 31.3 g (0.102 mol) 78%.

1.3 4-AcetyloxAmidinobenzylamine×HCl

5 mmol of Boc-4 acetyloxamidinobenzylamide are dissolved in 20 ml of 1 N HCl in glacial acetic acid and left to stand at room temperature for 45 min. The solution is then substantially concentrated in vacuo, and the product is precipitated with dry diethyl ether, filtered off with suction, again washed with fresh ether and dried in vacuo. Since reaction was quantitative, the product was employed without further purification for the next synthesis step.

1.4 Boc-Glu(OBzl) 4-Acetyloxamidinobenzylamide

Coupling of Boc-Glu(OBzl)-OH (Orpegen, Heidelberg) to 4-acetyloxamidinobenzylamine×HCl took place in accordance with Fréot et al. (Tetrahedron 47, 259 ff., 1991). For this, 2.27 g (9.3 mmol) of 4-acetyloxamidinobenzylamine×HCl and 3.138 g g (9.3 mmol) of Boc-Glu(OBzl)-OH were dissolved in approx. 25 ml of DMF. At 0° C., 4.84 g (9.3 mmol) of PyBOP and 3.878 ml (27.9 mmol) of TEA were added, and the pH was adjusted to 9 with TEA. Stirring at room temperature for 1 h was followed by concentration in vacuo, taking up in ethyl acetate and acidic, basic and neutral washings, 3 times each, drying with Na₂SO₄ and concentration in vacuo.

Yield: 4.1 g (7.8 mmol) 84%.

1.5 H-Glu(OBzl) 4-Acetyloxamidinobenzylamide×HCl

4.1 g (7.8 mmol) of Boc-Glu(Bzl) 4-acetyloxamidinobenzylamide were dissolved in 100 ml 1 N HCl in glacial acetic acid and left to stand at room temperature for 45 min. This was followed by substantial concentration in vacuo and precipitation with dry diethyl ether, and then filtration off with suction, and the product was again washed with fresh ether. After the product had been dried in vacuo it was employed without further purification for the synthesis in section 1.7.

1.6 Benzylsulfonyl-D-Ser(tBu)-OH

525 mg (3.257 mmol) of H-D-Ser(tBu)-OH and 1.187 ml (6.824 mmol) of DIEA were dissolved in 75 ml of 50% acetonitrile. Then 591 mg (3.102 mmol) of benzylsulfonyl chloride were added, and the mixture was stirred at room temperature for 12 h. It was concentrated in vacuo, taken up in ethyl acetate and subjected to acidic and neutral washings, 3 times each. Drying over sodium sulfate was followed by concentration in vacuo. Yield: 743 mg (2.357mmol) 76%.

1.7 Benzylsulfonyl-D-Ser(tBu)-Glu(OBzl) 4-acetyloxamidinobenzylamide

136 mg (0.433 mmol) of benzylsulfonyl-D-Ser(tBu)-OH and 194 mg (0.433 mmol) of H-Glu(OBzl) 4-acetyloxamidinobenzylamide×HCl were dissolved in 5 ml of abs. DMF. While cooling in ice, 225 mg (0.433 mmol) of PyBOP and 230 μl (1.32 mmol) of DIEA were added. After 2 h, the mixture was concentrated in vacuo, taken up in ethyl acetate and subjected to acidic, basic and neutral washings, 3 times each. Drying over sodium sulfate was followed by concentration in vacuo and hydrogenation without further workup as in section 1.8.

Yield: 242 mg (0.342 mmol) 79%.

1.8 Benzylsulfonyl-D-Ser(tBu)-Glu 4-amidinobenzylamide×TFA

242 mg (0.342 mmol) of Bzls-D-Ser(tBu)-Glu(OBzl) 4-acetyloxamidinobenzylamide were dissolved in 30 ml of 90% acetic acid. Then, under argon, 20 mg of 10% palladium on activated carbon were added. Argon was replaced by a hydrogen atmosphere, and the mixture was hydrogenated while stirring for 24 h. The catalyst was filtered off, the filtrate was concentrated in vacuo, and the product was purified by preparative reversed-phase HPLC (acetonitrile/H₂O, 0.1% trifluoroacetic acid, elution at 34.9% acetonitrile).

### EXAMPLE 2

### Inhibition of F Xa by Selected Acylated Amidinobenzylamine Compounds

| R₅ | R₄ configuration | R₄ | R₃ | X-R₂ | Y-R₁ | Kᵢ (μM) |
|---|---|---|---|---|---|---|
| Bzl—SO₂ | D | CH₂—O—tBu | H | CH₂ | CH₂ | 0.050 |
| Bzl—SO₂ | D | CH₂—O—tBu | H | CH—CH₂—COOH | CH₂ | 1.2 |
| Bzl—SO₂ | D | CH₂—O—tBu | H | CH—(CH₂)₂—COOH | CH₂ | 0.25 |

Determination of the Inhibitory Effect

The inhibitory effect was determined by incubating 200 μl of Tris buffer (0.05 M, 0.154 M NaCI, 5% ethanol, pH 8.0; contains the inhibitor), 25 μl of substrate (Moc-D-Nle-Gly-Arg-pNA in H₂O; Pentapharm Ltd., Basel, Switzerland) and 50 μl of F Xa (bovine, Diagnostic Reagents Ltd, Thame, GB) at 25° C. After 3 min, the reaction was stopped by adding 25 μl of acetic acid (50%), and the absorption at 405 nm was determined using a microplate reader (MR 5000, Dynatech, Denkendorf, Germany). The Kᵢ values were found by the method of Dixon (Biochem. J. 55, 170-171, 1953) by linear regression using a computer program. The Kᵢ values are the average of at least three determinations.

### EXAMPLE 3

Elimination after i.v. Administration of 1 mg/kg Body Weight of Derivatives of Benzylsulfonyl-D-Ser(tBu)-Gly 4-amidinobenzylamide with Ala, Asp or Glu in the P2 Position to Rats

[image]

Animal Experiments

Female Wistar rats (body weight 240-300 g) were anesthetized (ethylurethane 2.5 g/ml in NaCl, 0.5 ml/100 g rat), followed by dissection of the carotid artery located in the neck. A catheter fixed in this vessel made it possible to take blood at fixed times. The volume administered was 0.5 ml, and 0.9% NaCl was employed as administration solution. 500 μl blood samples (mixed in the ratio 19+1 with 1.04 M sodium citrate) were taken at the following times: 2, 5, 15, 30, 45, 60, 90, 120, 150, 180, 210, 240 and 270 min. The resulting blood loss was compensated with 500 μl of 0.9% NaCl solution immediately after taking the sample. Citrated plasma was obtained by centrifuging the blood at 1200*g for 10 min. The concentration of the active ingredients in the plasma was found by HPLC.

### EXAMPLE 4

### Inhibition of Factor Xa by Inhibitors of the General Structure According to Formula I

| No. | R₅ | P₂ | P₁ | NH-YR₁-V-U-Z | Kᵢ (μM) |
|---|---|---|---|---|---|
| 1. | 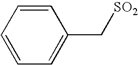 | D-Phe(3- | Gly | 4-Amba | 0.0065 |
| 2. | 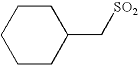 | D-Arg | Gly | 4-Amba | 0.0067 |
| 3. | 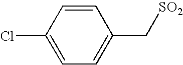 | D-Ser(tBu) | Gly | 4-Amba | 0.014 |
| 4. | 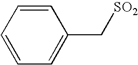 | D-Phe | Gly | 4-Amba | 0.026 |
| 5. | 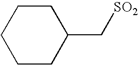 | D-Ser(tBu) | Ser | 4-Amba | 0.027 |
| 6. | 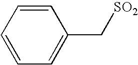 | D-Cha | Glu | 4-Amba | 0.028 |
| 7. | 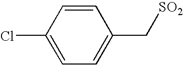 | D-Ser(tBu) | Gly | 4-Amba | 0.029 |
| 8. | 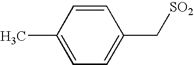 | D-Ser(tBu) | Gly | 4-Amba | 0.034 |
| 9. | 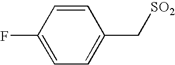 | D-Ser(tBu) | Gly | 4-Amba | 0.036 |
| 10. | 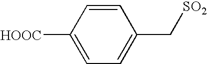 | D-Ser(tBu) | Gly | 4-Amba | 0.053 |
| 11. | 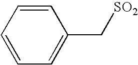 | D-Ser(tBu) | Ser | 4-Amba | 0.054 |
| 12. | 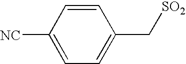 | D-Ser(tBu) | Gly | 4-Amba | 0.065 |
| 13. | 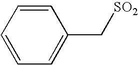 | D-Cha | Lys | 4-Amba | 0.067 |
| 14. | 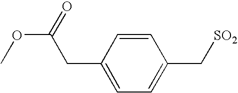 | D-Ser(tBu) | Gly | 4-Amba | 0.07 |
| 15. | 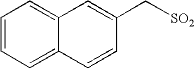 | D-Ser(tBu) | Gly | 4-Amba | 0.078 |
| 16. | 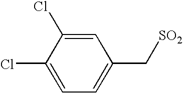 | D-Ser(tBu) | Gly | 4-Amba | 0.083 |
| 17. | 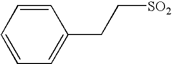 | D-Ser(tBu) | Gly | 4-Amba | 0.088 |
| 18. | 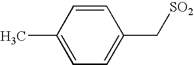 | D-Ser(tBu) | Ala | 4-Amba | 0.12 |
| 19. | 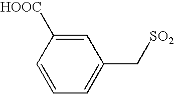 | D-Ser(tBu) | Gly | 4-Amba | 0.13 |
| 20. | 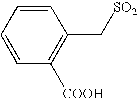 | D-Ser(tBu) | Gly | 4-Amba | 0.14 |
| 21. | 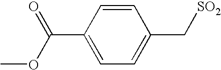 | D-Ser(tBu) | Gly | 4-Amba | 0.16 |
| 22. | 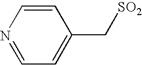 | D-Ser(tBu) | Gly | 4-Amba | 0.17 |
| 23. | 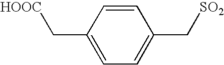 | D-Ser(tBu) | Gly | 4-Amba | 0.17 |
| 24. | 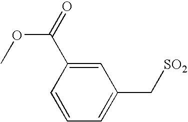 | D-Ser(tBu) | Gly | 4-Amba | 0.18 |
| 25. | 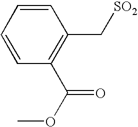 | D-Ser(tBu) | Gly | 4-Amba | 0.18 |
| 26. | 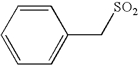 | D-Phe(4- Amidino) | Gly | 4-Amba | 0.26 |
| 27. | 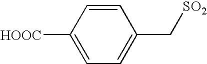 | D-Ser(tBu) | Ser | 4-Amba | 0.27 |
| 28. | 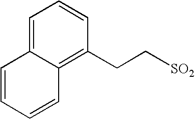 | D-Ser(tBu) | Gly | 4-Amba | 0.28 |
| 29. | 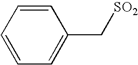 | D-Phe(4-CN) | Gly | 4-Amba | 0.30 |
| 30. | 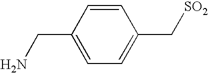 | D-Ser(tBu) | Gly | 4-Amba | 0.35 |
| 31. | 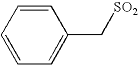 | D-Phe(4- Aminomethyl) | Gly | 4-Amba | 0.39 |
| 32. | 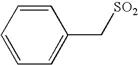 | D-His | Gly | 4-Amba | 0.67 |
| 33. | 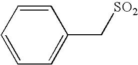 | D-Ser(tBu) | Gly | 4-Oxamidino- benzylamide | 26 |

### EXAMPLE 5

### Synthesis of Example 1: 3-(HOOC)Benzylsulfonyl-dSer(tBu)-Gly 4-amidinobenzylamide×TFA (No. 19. in the Table From Example 4)

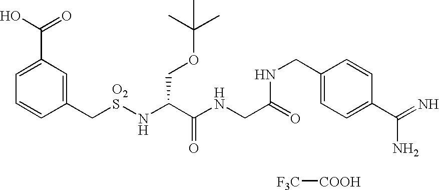

5a) Sodium 3-(COOMe)-benzylsulfonate

5 g (21.1 mmol) of methyl 3-(bromomethyl)benzoate (Lancaster) were suspended in 35 ml water and, after addition of 2.94 g (23.3 mmol) of Na₂SO₃, boiled under reflux for 8 h. The mixture was filtered hot and the water was concentrated in vacuo until crystallization started. The mixture was stored in a refrigerator overnight, and then the crystals were filtered off with suction and again recrystallized from water. The crystals were filtered off with suction and dried in vacuo.

Yield: 3.9 g (15.46 mmol) HPLC: 22.3% B

5b) 3-(COOMe)-Benzylsulfonyl chloride

2.5 g (9.91 mmol) of sodium 3-(COOMe)-benzylsulfonate were moistened with approx. 10 ml of phosphoryl chloride, mixed with 2.27 g (10.9 mmol) of PCl₅ and stirred in an ice bath for 15 minutes. The mixture was then heated at 80° C for 4 h. The mixture was subsequently poured onto ice and vigorously stirred for 30 min, and the product was deposited in the form of white crystals on the ice. After the ice had partially thawed, the mixture was filtered through a frit, and the remaining product/ice mixture was washed several times with water. The remaining crystals were dried in vacuo.

Yield: 1.6 g (6.43 mmol) 65% (white crystals)

5c) 3-(COOMe)-Benzylsulfonyl-dSer(tBu)-OH

0.75 g (4.65 mmol) of H-dSer(tBu)-OH (Bachem) were suspended in 60 ml of dry DCM and mixed with 1.23 ml (9.765 mmol) of trimethylsilyl chloride and 1.7 ml (9.765 mmol) of DIEA. The mixture was boiled under reflux for 1.0 h and then cooled in an ice bath. Subsequently, 1.27 g (5.12 mmol) of 3-(COOMe)-benzylsulfonyl chloride and 1.04 ml (6 mmol) of DIEA were added in several portions over the course of 30 min. The mixture was stirred while cooling in ice for a further 15 min and then at room temperature for 3 h. The solvent was removed in vacuo, and the residue was dissolved in water (adjusted to pH 8.5-9 with 1 N NaOH) and extracted 2× with ether. The aqueous phase was acidified with 5% KHSO₄ solution and extracted 3× with ethyl acetate. The combined ethyl acetate phases were washed 3× each with 5% KHSO₄ solution and NaCl-saturated solution and dried with Na₂SO₄. The solvent was then removed in vacuo.

Yield: 1.3 g (3.48 mmol of solid), HPLC: 51% B

5d) H-Gly 4-(Acetyloxamidino)benzylamide×HCl

2 g (5.49 mmol) of Boc-Gly 4-(acetyloxamidino)benzylamide (prepared as described in WO 01/96286 A2) were mixed with 30 ml of 1 N HCl in glacial acetic acid. The mixture was shaken occasionally. After 45 min, the solvent was concentrated somewhat, and the product was precipitated by adding diethyl ether, filtered off with suction on a frit, washed with ether and dried in vacuo.

Yield: 1.55 g (5.15 mmol), white solid

5e) 3-(COOMe)-Benzylsulfonyl-dSer(tBu)-Gly 4-(acetyloxamidino)benzylamide 1 g (2.68 mmol) of 3-(COOMe)-benzylsulfonyl-dSer(tBu)-OH and 0.84 g (2.8 mmol) of H-Gly 4-(acetyloxamidino)benzylamide×HCl were dissolved in 15 ml of DMF while stirring and cooling in ice, and 1.39 g (2.68 mmol) of PyBop and 1.26 ml (7.236 mmol) of DIEA were added. After 30 min, the ice bath was removed and stirring was continued at room temperature for 4 h. The DMF was concentrated in vacuo, and the remaining residue was dissolved in ethyl acetate and washed 3× each with 5% KHSO₄, NaCl-saturated water, saturated NaHCO₃ solution and again with NaCl-saturated water. The ethyl acetate phase was dried with Na₂SO₄, and then the solvent was removed in vacuo. The crude product was used without further purification for the next synthesis step.

Yield: 1.35 g (2.18 mmol) oil, HPLC: 47.89% B

5f) 3-(COOMe)-Benzylsulfonyl-dSer(tBu)-Gly 4-(amidino)benzylamid×acetate

1 g (1.61 mmol) of 3-(COOMe)-benzylsulfonyl-dSer(tBu)-Gly 4-(acetyloxamidino)benzylamide were dissolved in 65 ml of 90% acetic acid, mixed with 150 mg of catalyst (10% Pd on activated carbon) and hydrogenated with hydrogen overnight. The catalyst was filtered off, and the solvent was concentrated in vacuo. The remaining residue was mixed with toluene, and then the solvent was removed again in vacuo. This procedure was repeated once more. The remaining residue was used directly for the next reaction step.

Yield: 0.9 g (1.44 mmol) solid, HPLC: 39.75% B Approx. 50 mg of the crude product were purified by preparative reversed phase HPLC and lyophilized. MS: calculated 561.2 (monoisotopic), found 562.9 [M+H]⁺

5g) 3-(COOH)-Benzylsulfonyl-dSer(tBu)-Gly 4-(amidino)benzylamide×TFA 750 mg (1.2 mmol) of 3-(COOMe)-benzylsulfonyl-dSer(tBu)-Gly 4-(amidino)benzylamide×acetate were dissolved in 20 ml of methanol and 10 ml of water, and 4 ml of 1 N LiOH were added. The mixture was stirred overnight and, after approx. 15 h, neutralized (pH 6-7) with 5% KHSO₄, and the solvent was removed in vacuo. The crude product was purified by preparative reversed phase HPLC and lyophilized.

HPLC: 34.16% B (white solid) MS: calculated 547.21 (monoisotopic), found 548.3 [M+H]⁺

### EXAMPLE 6

### Benzylsulfonyl-dSer-Ser 4-amidinobenzylamide×TFA (No. 11 in the Table from Example 4)

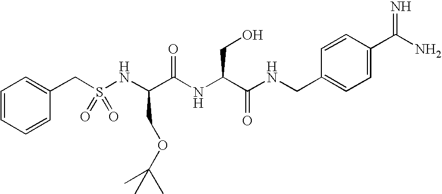

6a) Boc-Ser 4-(Acetyloxamidino)benzylamide

1 g (4.873 mmol) of Boc-Ser-OH were dissolved in 50 ml of DMF and, at −15° C., 0.536 ml (4.873 mmol) of NMM and 0.6335 ml (4.873 mmol) of BICC were added. The mixture was stirred at −15° C. for 10 min, and then 1.187 g (4.873 mmol) of 4-(acetyloxamidino)benzylamine×HCl (prepared as described in WO 01/96286 A2) and 0.536 ml (4.873 mmol) of NMM were added. After 20 min, a further 0.15 ml of NMM was added to the mixture. The mixture was stirred at −15° C. for a further hour and at room temperature overnight. The solvent was removed in vacuo, and the mixture was taken up in a large quantity of ethyl acetate and washed 1× with a little sat. NaHCO₃ solution and 2× with a little NaCl-saturated water and dried with Na₂SO₄. The solvent was removed in vacuo.

Yield: 1.2 g of white foam, HPLC: 29.9% B

6b) H-Ser 4-(Acetyloxamidino)benzylamide×TFA

1.1 g of Boc-Ser 4-(acetyloxamidino)benzylamide were mixed with 2 ml of water and 18 ml of TFA. After stirring at room temperature for 1 h, the product was precipitated by adding dry diethyl ether and was filtered off with suction and washed again with diethyl ether. The product was dried in vacuo.

Yield: 0.85 g of white solid, HPLC: 15.42% B

6c) Bzls-dSer(tBu)-Ser 4-(acetyloxamidino)benzylamide

0.2 g (0.634 mmol) of Bzls-dSer(tBu)-OH and 0.2097 g (0.634 mmol) of H-Ser 4-(acetyloxamidino)benzylamide×TFA were dissolved in 10 ml of DMF and, at 0° C., 0.329 g (0.634 mmol) of PyBop and 329 μl of DIEA were added. The mixture was stirred at 0° C. for 30 min and at room temperature for a further 4 h. The solvent was removed in vacuo, and the residue was taken up in a large quantity of ethyl acetate and washed 2× each with little volume of 5% KHSO₄, NaCl-saturated water, saturated NaHCO₃ solution and again with NaCl-saturated water and then dried with Na₂SO₄. The solvent was removed in vacuo. An oily crude product remained and was employed directly for the next synthesis step.

Yield: 0.31 g of oil, HPLC: 42.97% B

6d) Bzls-dSer(tBu)-Ser 4-(amidino)benzylamide×TFA

200 mg (0.29 mmol) of Bzls-dSer(tBu)-Ser(Bzl) 4-(acetyloxamidino)benzylamide were dissolved in 100 ml of 90% acetic acid, and 50 mg of catalyst (10% Pd/C) were added thereto. The mixture was hydrogenated with hydrogen under atmospheric pressure at room temperature for 6 h. The catalyst was then filtered off, the solvent was concentrated in vacuo, and the product was purified by preparative HPLC and lyophilized.

Yield: 75 mg (white solid), HPLC: 34.7% B. MS: calculated 533.23 (monoisotopic), found 534.5 [M+H]+

### EXAMPLE 7

### 4-(Aminomethyl)benzylsulfonyl-dSer(tBu)-Gly 4-amidinobenzylamide×2 TFA

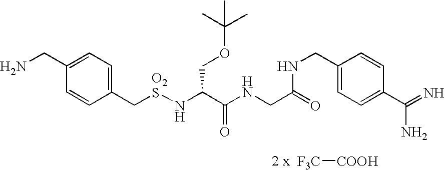

7a) Sodium 4-cyanobenzylsulfonate

30 g (153 mmol) of 4-cyanobenzyl bromide (Aldrich) were suspended in 150 ml of water and, after addition of 21.2 g (168.3 mmol) of Na2SO3 boiled under reflux for 8 h. The mixture was filtered hot and the water was concentrated somewhat in vacuo. The mixture was stored in a refrigerator overnight for crystallization, and then the crystals were filtered off with suction and again recrystallized from water. The crystals were filtered off with suction and dried in vacuo.

Yield: 17.1 g (78 mmol), HPLC: 18.24% B

7b) 4-Cyanobenzylsulfonyl chloride

5 g (22.83 mmol) of sodium 4-cyanobenzylsulfonate were moistened with approx. 20 ml of phosphoryl chloride and mixed with 5.2 g (25.11 mmol) of PCl5 and stirred while cooling in ice for 15 min. The mixture was subsequently heated at 80° C. for 4 h. The mixture was then poured onto ice and vigorously stirred for 30 min, while the product was deposited as white solid on the ice. After the ice had partially thawed, the mixture was filtered through a frit, and the remaining product/ice mixture was washed several times with water. The remaining crystals were dried in vacuo and used directly for the next synthesis step.

Yield: 3.4 g (15.76 mmol)

7c) 4-Cyanobenzylsulfonyl-dSer(tBu)-OH

1 g (6.2 mmol) of H-dSer(tBu)-OH (Bachem) were suspended in 50 ml of dry DCM, and 1.65 ml (13 mmol) of trimethylsilyl chloride and 2.26 ml (13 mmol) of DIEA were added. The mixture was boiled under reflux for 1 h and cooled in an ice bath. Then 1.47 g (6.82 mmol) of 4-cyanobenzylsulfonyl chloride and 1.19 ml (6.82 mmol) of DIEA were added over the course of 30 min. The mixture was stirred while cooling in ice for a further 15 min and then at room temperature for a further 3 h. The solvent was removed in vacuo, and the residue was dissolved in water (adjusted to pH 8.5-9 with 1 N NaOH) and extracted 2× with ether. The aqueous phase was then acidified with 5% KHSO₄ solution and extracted 3× with ethyl acetate. The combined ethyl acetate phase was washed 3× each with 5% KHSO4 solution and NaCl-saturated solution and dried with Na₂SO₄. The solvent was removed in vacuo.

Yield: 1.4 g (4.11 mmol of solid), HPLC: 48.89% B

7d) 4-Cyanobenzylsulfonyl-dSer(tBu)-Gly 4-(acetyloxamidino)benzylamide

1 g (2.94 mmol) of 4-cyanobenzylsulfonyl-dSer(tBu)-OH and 0.884 g (2.94 mmol) of H-Gly 4-(acetyloxamidino)benzylamide×HCl (see Example 1d) were dissolved in 15 ml of DMF while stirring and cooling in ice, and 1.53 g (2.94 mmol) of PyBop and 1.38 ml (7.94 mmol) of DIEA were added. After 30 min, the ice bath was removed and the mixture was stirred at room temperature for a further 4 h. The DMF was concentrated in vacuo, and the remaining residue was dissolved in ethyl acetate and washed 3× each with 5% KHSO4, NaCl-saturated water, saturated NaHCO3 solution and again with NaCl-saturated water and dried with Na2SO4. The solvent was removed in vacuo. The crude product was used without further purification for the next synthesis step.

Yield: 1.4 g (2.386 mmol) of oil, HPLC: 46.05% B

7e) 4-Cyanobenzylsulfonyl-dSer(tBu)-Gly 4-(amidino)benzylamide×acetate

1 g (1.7 mmol) of 4-cyanobenzylsulfonyl-dSer(tBu)-Gly 4-(acetyloxamidino)benzylamide were dissolved in 70 ml of 90% acetic acid, mixed with 150 mg of catalyst (10% Pd on activated carbon) and hydrogenated with hydrogen for 5 h. The catalyst was filtered off and the solvent was concentrated. The remaining residue was mixed with toluene and then the solvent was removed in vacuo. This procedure was repeated again. The remaining residue was used directly for the next reaction step.

Yield: 0.85 g (1.44 mmol as acetate salt) of solid HPLC: 37.55% B Approx. 60 mg of this crude product were purified by preparative HPLC. MS: calculated 528.2 (monoisotopic), found 530.1 [M+H]+

7f) 4-Aminomethylbenzylsulfonyl-dSer(tBu)-Gly 4-(amidino)benzylamide×2 TFA

200 mg of 4-cyanobenzylsulfonyl-dSer(tBu)-Gly 4-(amidino)benzylamide×acetate crude product were dissolved in 50 ml of 90% acetic acid and 5 ml of 1 N HCl, mixed with 40 mg of catalyst (10% Pd on activated carbon) and hydrogenated with hydrogen at 40° C. overnight. The catalyst was filtered off and the solvent was concentrated in vacuo. The remaining residue was purified by preparative reversed phase HPLC.

Yield: 75 mg (as 2× TFA salt) solid HPLC: 26.05% B MS: calculated 532.25 (monoisotopic), found 533.7 [M+H]+

### EXAMPLE 8

### Benzylsulfonyl-dPhe(3-amidino)-Gly 4-(amidino)benzylamide×2 TFA

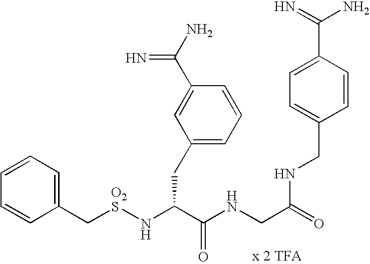

8a) Benzylsulfonyl-dPhe(3-CN)-OH

1 g (4.42 mmol) of H-dPhe(3-CN)-OH×HCl were dissolved in a mixture of 40 ml of dioxane and 10 ml of water and the pH was adjusted to pH 8-9 by adding DIEA. The mixture was cooled in an ice bath and, over a period of 3 h, a total of 1.265 g (6.63 mmol) was added in several portions, adjusting the pH to 8-9 by addition of DIEA. After 1 h, the ice bath was removed and the mixture was stirred at RT overnight. The solvent was removed in vacuo, and the residue was taken up in ethyl acetate and 5% KHSO4 solution. The ethyl acetate phase was washed 3× with 5% KHSO₄ solution and 3× with NaCl-saturated aqueous solution. The ethyl acetate phase was dried with Na₂SO₄, and the solvent was removed in vacuo. The product was crystallized from ethyl acetate.

Yield: 1.6 g (white solid), HPLC at 45.8% B8

8b) Benzylsulfonyl-dPhe(3-acetyloxamidino)-OH

800 mg (2.32 mmol) of benzylsulfonyl-dPhe(3-CN)-OH were dissolved in 30 ml of methanol, and 280 mg (4 mmol) of hydroxylamine×HCl and 0.63 ml (3.6 mmol) of DIEA were added. The mixture was boiled under reflux for 5 h and stirred at RT overnight. The solvent was removed in vacuo, and the residue was dissolved in 30 ml of acetic acid. 665 μl (7 mmol) of acetic anhydride were added, and the mixture was stirred at RT for 30 min. The solvent was then removed in vacuo, and the residue was dissolved in ethyl acetate and washed 2× with 5% KHSO₄ solution and 3× with NaCl-saturated aqueous solution. The-ethyl acetate phase was dried with Na₂SO₄, and the solvent was removed in vacuo. Yield: 805 mg (oil), HPLC at 38.5% B

8c) Benzylsulfonyl-dPhe(3-acetyloxamidino)-Gly 4-(acetyloxamidino)benzylamide

100 mg (0.24 mmol) of benzylsulfonyl-dPhe(3-acetyloxamidino)-OH and 75 mg (0.25 mmol) of H-Gly 4-(acetyloxamidino)benzylamide×HCl (see Example 1d) were dissolved in 5 ml of DMF while stirring and cooling in ice, and 125 mg (0.24 mmol) of PyBop and 125 μl of DIEA were added. After 30 min, the ice bath was removed and the mixture was stirred at room temperature for a further 3 h. The DMF was concentrated in vacuo, and the remaining residue was dissolved in ethyl acetate and washed 3× each with 5% KHSO4, NaCl-saturated water, saturated NaHCO3 solution and again with NaCl-saturated water and dried with Na2SO4. The solvent was removed in vacuo. The crude product was used without further purification for the next synthesis step.

Yield: 1.43 mg yellowish oil, HPLC: 38.3% B

8d) Benzylsulfonyl-dPhe(3-Amidino)-Gly 4-(amidino)benzylamide

100 mg of benzylsulfonyl-dPhe(3-acetyloxamidino)-Gly 4-(acetyloxamidino)benzylamide were dissolved in 20 ml of glacial acetic acid, and 20 mg of catalyst (10% Pd/C) were added. The mixture was hydrogenated under a hydrogen atmosphere overnight. The catalyst was filtered off, and the solvent was removed in vacuo. The product was purified by preparative HPLC.

Yield: 25 mg, HPLC at 24.6% B MS: calculated 549.22 (monoisotopic), found 550.3 [M+H]+

Abbreviations used:
Ac acetyl
4-Amba 4-amidinobenzylamide
Boc tert-butyloxycarbonyl
Bzl benzyl
dCha d-βcyclohexylalanine
Dab α,γ-diaminobutyric acid
Dap α,β-diaminopropionic acid
DIEA diisopropylethylamine
DMF N,N-dimethylformamide
IBCC isobutyl chlorocarbonate
NMM N-methylmorpholine
PyBOP benzotriazol-1-yl-N-oxy-tris(pyrrolidino)phosphonium hexafluorophosphate
TEA triethylamine
TFA trifluoroacetic acid
THF tetrahydrofuran
tBu tert-butyl

1-15. (canceled)

16. A compound of the formula I 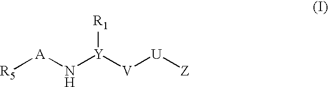
where
A is P₂-P₁ with 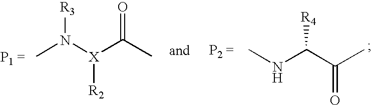
R₁is H or —(CH₂)ₐCOOR₆ with a=0, 1, 2, 3, 4, or 5, preferably with a=0, 1, or 2, where R₆ is a branched or unbranched alkyl radical having preferably 1 to 6 C atoms, in particular 1 to 3 C atoms, especially ethyl;
R₂ is —(CH₂)_{c}COOR₈ with c=1, 2, 3, or 4, where R₈ is H or a branched or unbranched alkyl radical having preferably 1 to 6 C atoms, in particular 1 to 3 C atoms, especially ethyl, or
—(CH₂)ₑ-guanidino, —(CH₂)ₑ-imidazole, or —(CH₂)ₑNHR₁₀ with e=1, 2, 3, 4, or 5, where R₁₀ is H, a branched or unbranched alkyl radical having 1-16, in particular 1-8, especially 1-3, C atoms or a substituted or unsubstituted aryl, heteroaryl, aralkyl, or heteroaralkyl radical, where the alkyl radical preferably has 1 to 16, in particular 1 to 8, especially 1 to 3, C atoms, and the aryl or heteroaryl radical preferably has 4 to 14, in particular 6 to 10, especially 6, C atoms and preferably 1 to 3 N as heteroatom; und
R₄ is a branched or unbranched alkyl radical having 1 to 8, preferably 1 to 3, C atoms, —(CH₂)_{f}OR₁₁, —(CH₂)_{f}SR ₁, —(CH₂)_{f}guanidino, —(CH₂)_{f}-imidazole, —(CH₂)_{f}—R₁₁ or —(CH₂)_{f}NHR₁₁ with f=1, 2, 3, 4, or 5, preferably 1 or 2, in particular 1, where R₁₁ is a branched or unbranched alkyl radical having 1 to 16, preferably 1 to 8, in particular 1-4 C atoms, especially tbutyl or a substituted or unsubstituted aryl, heteroaryl, aralkyl, or heteroaralkyl radical, where the alkyl radical preferably has 1 to 16, in particular 1 to 8, especially 1 to 3, C atoms, and the aryl or heteroaryl radical preferably has 4 to 14, in particular 6 to 10, especially 6, C atoms and preferably 1 to 3 N as heteroatom; where P2 in the structure A of the formula I is in the D configuration;
or
R₂ is an H; and
R₄ is —(CH₂)_{f}-guanidino, —(CH₂)_{f}-imidazole, or —(CH₂)_{f}R₁₁ with f=1, 2, 3, 4, or 5, preferably 1 or 2, in particular 1, where R₁₁ is a substituted or unsubstituted aryl or heteroaryl radical which preferably has 4 to 14, in particular 6 to 10, especially 6, C atoms and preferably 1 to 3 N as heteroatom; where P2 in the structure A of the formula I is in the D configuration;
or
R₂ is a branched or unbranched alkyl radical having 1 to 8 C atoms, preferably having 1 to 3 C atoms, or —(CH₂)_{d}—OR₉ with d=1, 2, 3, or 4, where R₉ is H, or —(CH₂)ₑOR₁₀ or —(CH₂)ₑSR₁₀, with e=1, 2, 3, 4, where R₉ is H, or —CH₂)ₑOR₁₀ or (CH₂)ₑSR₁₀, with e=1, 2, 3, 4, or 5, where R₁₀ is H, a branched or unbranched alkyl radical having 1-16, in particular 1-8, especially 1-3, C atoms or a substituted or unsubstituted aryl, heteroaryl, aralkyl or heteroaralkyl radical, where the alkyl radical preferably has 1 to 16, in particular 1 to 8, especially 1 to 3, C atoms, and the aryl or heteroaryl radical preferably has 4 to 14, in particular 6 to 10, especially 6, C atoms and preferably 1 to 3 N as heteroatom; and
R₄ is —(CH₂)_{f}-guanidino, —(CH₂)_{f}-imidazole or —(CH₂)_{f}—R₁₁ with f=1, 2, 3, 4, or 5, preferably 1 or 2, in particular 1, where R₁₁ is a substituted or unsubstituted or heteroaryl radical which preferably has 4 to 14, in particular 6 to 10, especially 6, C atoms and preferably 1 to 3 N as heteroatom; where P2 in the structure A of the formula I is in the D configuration;
R₃ is H or —(CH₂)_{b}R₇ with b=1, 2, 3, 4, 5, 6, 7, or 8, preferably with b=2 or 3, where R₇ is H, a branched or unbranched alkyl radical having 1 to 10 C atoms, preferably having 1 to 3 C atoms, or a charged radical, preferably a —(CH₂)ⱼCOOR₁₃, —(CH₂)ⱼSO₂R₁₃, —(CH₂)ⱼNH₂, —(CH₂)ⱼ-amidino, —(CH₂)ⱼ-hydroxyamidino, or —(CH₂)ⱼ-guanidino group with j=0, 1 or 2, where R₁₃ is H or an alkyl radical having preferably 1 to 6 C atoms, in particular 1 to 4, especially ethyl;
R₅ is —SO₂R₁₂, where R₁₂ is a substituted or unsubstituted aralkyl or heteroalkyl radical, preferably benzyl,
where R₅ may be modified with a charged or uncharged group, preferably a —(CH₂)ⱼCOOR₁₃, —(CH₂)ⱼSO₂R₁₃, —(CH₂)ⱼNH₂, —(CH₂)ⱼ-amidino, —(CH₂)ⱼ-hydroxyamidino, or —(CH₂)ⱼ-guanidino group with j=0, 1, or 2, where R₁₃ is H or an alkyl radical having preferably 1 to 6 C atoms, in particular 1 to 4, especially ethyl;
U is a phenyl or cyclohexyl radical;
a heterophenyl or heterocyclohexyl radical having preferably at least one N, S, or O as heteroatom, in particular pyridine, piperidine, or pyrimidine, or is a thiophene radical;
V is (CH₂)ₙ with n=0, 1, 2 or 3, preferably 0;
X is N or CH, preferably CH;
Y is N or (CH)ₘ with m=0 or 1, preferably CH;
Z occurs in the 3 or 4 position and is an aminomethyl, a guanidino function, or an amidino group, 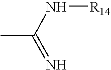
where R₁₄ is H, OH, NH₂, —COR₁₅ or —COOR₁₅, where R₁₅ is a branched or unbranched alkyl radical having 1 to 16, preferably 1 to 8, in particular 1 to 4, especially 1 to 2, C atoms or a substituted or unsubstituted aryl or heteroaryl, aralkyl or heteroaralkyl radical, where the alkyl radical preferably has 1 to 16, in particular 1 to 8, especially 1 to 4 and particularly preferably 1 to 2, C atoms and the aryl or heteroaryl radical preferably has 4 to 14, in particular 6 to 10, especially 6, C atoms and preferably 1 to 3 N as heteroatom;
characterized in that one or more charged radicals preferably derived from —COOH, —CH(COOH)₂, —SO₂H, NH₂, an amidino, hydroxyamidino, amidrazono, or guanidino group are present in the radicals R₁, R₂, R₃ or R₅;
or a compound of the formula I in the form of a prodrug or in the form of its salt.

17. The compound as claimed in claim 16, where U is substituted at 1, 2, or 3 positions preferably by a halogen, in particular fluorine or chlorine, or a methyl, ethyl, propyl, methoxy, ethoxy, or propoxy radical.

18. The compound as claimed in claim 16, where a carboxyl group is present protected as ester, preferably as ethyl ester, and is converted into a carboxyl group in the manner of a prodrug only after intake in the body.

19. The compound as claimed in claim 16, where R₉ in this case is an alkylcarbonyl, aralkylcarbonyl, alkyloxycarbonyl, or aralkyloxycarbonyl radical, where the alkyl radical preferably has 1 to 6, in particular 1 to 4, C atoms and the aryl radical preferably has 5 to 8, in particular 6, C atoms; and where R₉ is converted into a carboxyl group in the manner of a prodrug only after intake in the body.

20. The compound as claimed in claim 16, characterized in that P₂ in structure A of the formula I is derived from one of the following amino acids in the D configuration: D-2,3-diaminopropionic acid, D-2,4-diaminobutyric acid, D-ornithine, D-citrulline, D-homocitrulline, D-norcitrulline, D-arginine, D-homoarginine, D-norarginine, D-4-guanidinophenylalanine, D-4-guanidinophenylhomoalanine, D-4-guanidinophenylglycine, D-3-guanidinophenylalanine, D-3-guanidinophenylhomoalanine, D-3-guanidinophenylglycine, D-4-amidinophenylalanine, D-4-amidinophenylhomoalanine, D-4-amidinophenylglycine, D-3-amidinophenylalanine, D-3-amidinophenylhomoalanine, D-3-amidinophenylglycine, D-4-aminomethylphenylalanine, D-4-aminomethylphenylhomoalanine, D-4-aminomethylphenylglycine, D-3-aminomethylphenylalanine, D-3-aminomethylphenylhomoalanine, D-3-aminomethylphenylglycine, D-4-guanidinomethylphenylalanine, D-4-guanidinomethylphenylhomoalanine, D-4-guanidinomethylphenylglycine, D-3-guanidinomethylphenylalanine, D-3-guanidinomethylphenylhomoalanine, D-3-guanidinomethylphenylglycine, D-4-piperidinylalanine, D-4-piperidinylhomoalanine, D-4-piperidinylglycine, D-4-N-(amidino)piperidinylalanine, D-4-N-(amidino)piperidinylhomoalanine, D-4-N-(amidino)piperidinylglycine, D-3-piperidinylalanine, D-3-piperidinylhomoalanine, D-3-piperidinylglycine, D-3-amidinopiperidinylalanine, D-3-amidinopiperidinylhomoalanine, D-3-amidinopiperidinylglycine, D-4-aminocyclohexylalanine in cis or trans, D-4-aminocyclohexylhomoalanine in cis or trans, D-4-aminocyclohexylglycine in cis or trans, n-butylamidinoglycine, n-pentylamidinoglycine, n-propylamidinoglycine, D-alanine(3-(1-N-piperazinyl), or D-homoalanine(3-(1-N-piperazinyl).

21. The compound as claimed in claim 16, characterized in that P₂ in the structure A of the formula I is derived from one of the following amino acids in the D configuration: D-canavanine, D-homocanavanine, D-norcanavanine, 2-amino-4-amidinohydrazonobutyric acid, 2-amino-3-amidinohydrazonopropionic acid, 2-amino-5-amidinohydrazonopentanoic acid, 2-amino-4-(pyridin-4-ylamino)butyric acid, 2-amino-4-(pyridin-4-ylamino)propionic acid, 2-amino-4-(pyridin-4-ylamino)pentanoic acid, 4-imidazolylpropargylglycine, D-histidine, D-homohistidine, D-histidine-(1-methyl), D-homohistidine-(1-methyl), D-histidine-(3-methyl), D-homohistidine-(3-methyl), D-alanine(4-[5-2(-amino)imidazoyl], D-homoalanine(4-[5-2(-amino)imidazoyl], D-glycine(4-[5-2(-amino)imidazoyl], D-alanine(4-pyridyl), D-homoalanine(4-pyridyl), D-glycine(4-pyridyl), D-alanine(3-pyridyl), D-homoalanine(3-pyridyl), D-glycine(3-pyridyl), D-alanine(2-pyridyl), D-homoalanine(2-pyridyl), D-glycine(2-pyridyl), D-alanine(3-(2-pyrimidinyl), D-homoalanine(3-(2-pyrimidinyl), D-alanine(3-(5-pyrimidinyl), D-homoalanine(3-(5-pyrimidinyl), D-2-amino-3-(2-aminopyrimidin-5-yl)propionic acid, D-2-amino-4-(2-amino-pyrimidin-5-yl)butyric acid, D-alanine(3-(2-benzimidazolyl)), D-homoalanine(3-(2-benzimidazolyl)), D-alanine(3-(3-quinolinyl), D-homoalanine(3-(3-quinolinyl), D-tryptophan, D-homotryptophan, D-tryptophan substituted by aminoalkyl groups on the indole ring, D-homotryptophan substituted by aminoalkyl groups on the indole ring, D-2-amino-3-(6-aminopyridin-3-yl)propionic acid, D-2-amino-4-(6-aminopyridin-3-yl)butyric acid, D-2-amino-3-(6-amino-2-methylpyridin-3-yl)propionic acid, D-2-amino-4-(6-amino-2-methylpyridin-3-yl)butyric acid, D-2-amino-3-(6-amino-2,4-dimethylpyridin-3-yl)propionic acid, D-2-amino-4-(6-amino-2,4-dimethylpyridin-3-yl)butyric acid, D-4-hydroxyamidinophenylalanine, D-4-hydroxyamidinophenylhomoalanine, D-4-hydroxyamidinophenylglycine, D-3-hydroxyamidinophenylalanine, D-3-hydroxyamidinophenylhomoalanine, D-3-hydroxyamidinophenylglycine, D-4-aminophenylalanine, D-4-aminophenylhomoalanine, D-4-aminophenylglycine, D-3-aminophenylalanine, D-3-aminophenylhomoalanine, and D-3-aminophenylglycine.

22. A compound of the formula I, characterized in that the compound has the following structure: 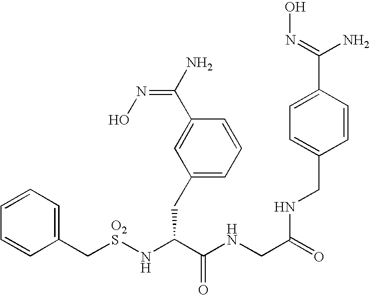
where the hydroxyamidino groups present in the structure are converted into the analogous amidino groups in the manner of a prodrug only after intake in the body, resulting in the inhibitor structure with inhibitory activity.

23. The compound as claimed in claim 16, characterized in that the substituent on the substituted aryl, heteroaryl, aralkyl, or heteroaralkyl radical is a halogen, preferably fluorine, chlorine or bromine, in particular fluorine or chlorine.

24. The compound as claimed in claim 16, characterized in that the compounds are preferably in the form of salts, preferably with mineral acids, preferably as hydrochlorides, or preferably as salts with suitable organic acids.

25. The compound as claimed in claim 24, characterized in that preferred salts of mineral acids are also sulfates, and suitable organic acids are, for example, acetic acid, formic acid, methylsulfonic acid, succinic acid, malic acid, or trifluoroacetic acid, with preferred salts of organic acids being acetates.

26. A method for preparing a compound as claimed in claim 16, the method comprising sequentially coupling the appropriate amino acids onto a 4-acetyloxamidinobenzylamine, with the N-terminal amino acid either already carrying the R₅ radical or the latter subsequently being linked thereto.

27. A medicament comprising a compound as claimed in claim 16 and pharmaceutically suitable excipients and/or additives.

28. The medicament as claimed in claim 27, where the medicament is employed in the form of a tablet, a coated tablet, a capsule, a pellet, a suppository, a solution, in particular a solution for injection or infusion, eyedrops, nosedrops, eardrops, a syrup, a capsule, an emulsion or suspension, a pessary, stick, aerosol, dusting powder, a paste, cream, or ointment.

29. A method of treating or preventing a cardiovascular disorder or a thromboembolic event, said method comprising administering to a patient a compound as claimed in claim 16, in particular in oral, subcutaneous, intravenous or transdermal form.
